(19) United States
(12) Patent Application Publication
(10) Pub. No.: US 2019/0091494 A1
(43) Pub. Date: Mar. 28, 2019

(54) PEPTIDES FOR HAIR GROWTH

(71) Applicant: Clinical Stem Cells Pty Ltd, Alexandria (AU)

(72) Inventor: Edward Russell VICKERS, Alexandria (AU)

(73) Assignee: Clinical Stem Cells Pty Ltd, Alexandria (AU)

(21) Appl. No.: 16/085,904

(22) PCT Filed: Jun. 6, 2017
(86) PCT No.: PCT/AU2017/050551

§ 371 Date: Sep. 17, 2018

(30) Foreign Application Priority Data
Jun. 6, 2016 (AU) 2016902192
Jan. 25, 2017 (AU) 2017900229

(51) Int. Cl. A61Q 7/00; A61K 8/27; A61K 38/08; A61K 8/64; A61P 17/14; A61K 38/22

(52) U.S. Cl. CPC A61Q 7/00; A61K 8/27; A61K 38/08; A61K 2800/58; A61P 17/14; A61K 38/2292; A61K 8/64

(57) ABSTRACT

A method of activating inactive hair follicles by administering a composition including an effective amount of zinc thymulin to a subject. The method generates new hair growth and may be used to treat or prevent hair loss or, alternatively, to increase the quantity of hair in a subject that does not suffer from hair loss.

### FIELD OF THE INVENTION

The present invention relates to peptides, compositions and methods for improving hair growth, improving hair pigmentation, improving the quantity of hair that appears to be present and/or treating and/or preventing hair loss.

### BACKGROUND OF THE INVENTION

In many cultures, it is considered desirable to maintain the appearance of abundant hair in certain physiological regions, for example, on the scalp. Consequently, treatments for hair loss and products for improving the cosmetic appearance of hair have been developed. For example, there are products for temporary cosmetic hair volumising and hair thickening products.

A significant proportion of the population has “fine” hair with a thinner and less voluminous appearance than is desirable. The commercially available methods for improving the appearance of volume for fine hair include hair extensions (that is fixing exogenous hair to the endogenous hair) and topical products that expand the size of each hair shaft, for example, by layering fibre onto the hair or products that hold individual hairs apart thereby making the total volume of the hair appear larger. These methods for improving hair volume are temporary. They do not improve the quality of the person's endogenous hair and only temporarily alter how the hair appears.

Hair loss occurs in a large percentage of the adult human population and increases in prevalence with increasing age. Hair loss may occur in males and in females but is more prevalent in males. In the western population it is estimated that 50% of the male population have noticeable hair loss by 50 years of age. The most common form of hair loss in men is termed androgenetic alopecia (male pattern baldness). Most hair loss involves inactivation of hair follicles, that is, hair follicles cease to grow hair.

Normal human hair density varies between 100-300 follicles per square centimetre. Normal hair growth is approximately 1.25 cm/month. The hair growth cycle that occurs in active hair follicles includes several phases: anagen (a growth phase lasting 2-9 years), telogen (an intermediate rest phase of 1-4 months) and catagen (a hair loss phase of 2-4 weeks). Approximately 85% of the hair follicles are in anagen at any one time. During the catagen phase stem cells are stimulated to proliferate and produce a new hair follicle ready for the next anagen phase. This process is necessary to maintain hair growth.

A person may be losing hair yet still have normal hair density. For example, a person losing hair may have started with 300 follicles per square centimetre but now have 200 follicles per square centimetre. 200 follicles per square centimetre is still normal hair density.

As discussed above, a person suffering from hair loss can use products to improve the appearance of the volume of their hair. In addition, a few treatments for hair loss are available. Current treatments for hair loss include the FDA approved topical drugs finasteride and minoxidil, surgical hair transplants and cosmetic coverings using wigs. Minoxidil and finasteride can retard, that is, cease or lessen the extent of hair loss but neither drug generates new hair growth. Consequently, current topical treatments are limited to preventing further hair loss or lessening the speed of hair loss and cannot reverse hair loss that has already occurred. In addition, the retardation or lessening of hair loss will cease if treatment is ceased and the risk of side effects is significant. Further, minoxidil tends to only be effective on balding of the vertex or top of the head that is less than 10 cm in diameter.

The current hair loss treatments are invasive (ie surgical) or have a higher than desirable risk of side effects. There is no treatment available able to reactivate inactive hair follicles to generate new hair growth.

Thymulin is a nonapeptide with the amino-acid sequence pyrGlu-Ala-Lys-Ser-Gln-Gly-Gly-Ser-Asn (CAS 63958-90-7; molecular weight 858.85). It is produced by the thymus gland and is known to enhance the expression of a number of T-cell markers and functions in vivo. Thymulin is suspected to have a role in the communication between the neuroendocrine system and the immune system.

Thymic peptides including thymulin, thymosin alpha-1 and thymosin beta-4 have been suggested as treatments for hair loss that prevent hair shedding by catagen inhibition (see US 2011/0281802). The thymulin was suggested to avoid loss of hair by increasing the length of anagen phase and therefore increase the length of hair growth despite inhibiting hair shaft formation. There is nothing in US 2011/0281802 suggesting that thymulin can activate inactive hair follicles. In contrast, thymulin was found to inhibit hair shaft formation even in active hair follicles that entered catagen.

There is a need for treatments that improve hair growth rather than prevent further hair loss. There is also a need for treatments that reverse hair loss. There is a need for additional non-invasive hair loss treatment options that have less side effects than the current options. There is also a need for a method of improving the appearance of fine hair that is lasting and improves the quality of the endogenous hair.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a zinc thymulin metallo-peptide for activating inactive hair follicles. The invention further provides compositions containing the zinc thymulin metallo-peptide. The activation of inactive hair follicles optionally also improves hair growth, the apparent quantity of hair and/or treat and/or prevent hair loss. The zinc thymulin metallo-peptide optionally also improves endogenous hair pigmentation. For example, by stimulating melanogenesis in grey or greying hair.

The inventor of the present invention has demonstrated that the zinc thymulin metallo-peptide activates inactive hair follicles. In particular, the zinc thymulin metallo-peptide results in hair growth from hair shafts that had ceased growing hair. Consequently, the present invention increases the density of hair in a subject, that is, increases the number of hairs per square centimeter. In a person suffering from hair loss, the reactivation of inactive hair follicles so that follicles that were not growing hair start to grow hair not only prevents further hair loss but reverses hair loss that has already occurred.

The inventor of the present invention has observed that zinc thymulin is effective in the activation of inactive hair follicles whereas thymulin per se does not significantly activate inactive hair follicles.

In one aspect, the present invention provides a method of activating inactive hair follicles comprising administering a composition comprising an effective amount of zinc thymulin to a subject. The zinc thymulin is preferably exogenous. Zinc occurs in the body. Therefore, it is theoretically possible that, when thymulin per se is administered to a subject, some of the thymulin will complex with the zinc that naturally occurs in the body. Nevertheless, the results achieved when administering thymulin per se indicate that any complexing of the thymulin with naturally occurring zinc from the body is insufficient for the activation of inactive hair follicles. Therefore, application of exogenous zinc thymulin is preferred as a method of achieving administration of an effective amount of zinc thymulin.

The method may be a therapeutic method or a cosmetic method. The method optionally also increases hair density. Optionally, the method also increases the rate of hair growth. In some embodiments, the subject has a normal quantity of hair (ie normal hair density). For example, a subject with a normal quantity of hair optionally improves their hair growth or density by administration of zinc thymulin. Optionally, a subject with a normal quantity of hair administers zinc thymulin to prevent hair loss. Optionally, the subject suffers from hair loss. Where the subject suffers from hair loss, it is preferred that the hair loss is reversed. It is also preferred that the hair loss is treated/reversed by generating new hair growth (ie not only by preventing further hair loss). Where the subject suffers from hair loss, the activation of inactive hair follicles is a reactivation of hair follicles that were once active but were inactive prior to administration of the zinc thymulin. Alternatively, the present invention provides the use of an effective amount of zinc thymulin in the manufacture of a medicament for activating inactive hair follicles. In another alternative, the present invention provides a composition containing an effective amount of zinc thymulin for activating inactive hair follicles.

In another aspect, the present invention provides a method of reversing hair loss comprising administering a composition comprising an effective amount of zinc thymulin to a subject. Preferably, the zinc thymulin is exogenous. Preferably, the hair loss is reversed by generating new hair growth. The generation of new growth is preferred to be due to activation of inactive hair follicles or, more particularly, because hair follicles that were once active but were inactive prior to administration of the zinc thymulin have been reactivated. The method optionally also increases hair density. The method optionally also increases the rate of hair growth. Alternatively, the present invention provides the use of an effective amount of zinc thymulin in the manufacture of a medicament for reversing hair loss. In another alternative, the present invention provides a composition containing an effective amount of zinc thymulin for reversing hair loss. Other features of this aspect of the invention are as described above.

In a further aspect, the present invention provides a method of preventing hair loss by administering a composition comprising an effective amount of zinc thymulin to a subject and thereby increasing the number of active hair follicles or activating inactive hair follicles. Preferably, subject's hair growth and/or hair density is improved. Preferably, the zinc thymulin is exogenous. Alternatively, the present invention provides the use of an effective amount of zinc thymulin in the manufacture of a medicament for preventing hair loss. In another alternative, the present invention provides a composition containing an effective amount of zinc thymulin for preventing hair loss. Other features of this aspect of the invention are as described above.

In one aspect, the present invention provides a cosmetic method for improving the appearance of hair comprising administering a composition comprising an effective amount of zinc thymulin to a subject. Preferably, the method results in an appearance of a greater quantity of hair than before administration of the zinc thymulin. Preferably, the zinc thymulin is exogenous. Preferably, the number of active hair follicles is increased or inactive hair follicles are activated.

Alternatively, the present invention provides a method for increasing hair density in a subject that does not suffer from hair loss by administering a composition comprising an effective amount of zinc thymulin to the subject and thereby activating inactive hair follicles.

In another aspect, the present invention provides a method of improving the visual analog scale (VAS) hair assessment of a subject comprising administering a composition comprising an effective amount of zinc thymulin to a subject. Optionally, the improvement in the VAS hair assessment is statistically significant in a plurality of subjects that have received daily or twice daily administration of an effective amount of zinc thymulin for 4, 6 or 10 months. Optionally, the subject suffers from androgenetic alopecia. Optionally, the VAS hair assessment result improves by 1 to 3 units.

In another aspect, the present invention provides a method of improving the result of a hair growth index (HGI) hair assessment of a subject comprising administering a composition comprising an effective amount of zinc thymulin to a subject. The subject has an initial HGI hair assessment of absent, vellus or intermediate in a specific region before treatment and the assessment in that region optionally improved by 1 or 2 categories following 6 months of treatment.

An alternate method for improving the HGI hair assessment of a subject comprises categorising the percentage of the subject's hair in an absent, vellus, intermediate and terminal HGI category, administering a composition comprising an effective amount of zinc thymulin to the subject, reassessing the percentage of the subject's hair in an absent, vellus, intermediate and terminal HGI category following the administration to demonstrate a decreased percentage of absent hair regions. Optionally there is a decrease in both absent and vellus hair regions. Alternatively, there is an increase in terminal hair regions or an increase in both intermediate and terminal hair regions.

In a further aspect, the present invention provides a method of increasing melanogenesis by administering a composition comprising an effective amount of zinc thymulin to a subject and thereby increasing the number of hair follicles producing hair with natural pigment. Alternatively, the invention provides a method of increasing the number of hair follicles producing hair with natural pigment comprising administering an effective amount of zinc thymulin to a subject. The method optionally also activates inactive hair follicles. Optionally the activation of inactive hair follicles results in hair with melanin or increased melanin. Optionally, the method increases the number of hair follicles producing hair with colour (ie natural pigment).

In some embodiments of the invention, the subject suffers from male pattern baldness, hair loss following chemotherapy or radiation therapy, hair loss following significant weight loss, hair loss caused by a side effect of medication, hair loss caused by hormonal changes (such as menopause) or stress related hair loss. In alternate embodiments of the invention, the method improves hair growth following transgender surgery or when taking gender reassignment hormone treatments.

In some embodiments of the invention, the administration of zinc thymulin increases the number of active hair follicles by 5 to 5,000%, 5 to 3,000%, 5 to 1,000%, 5 to 500%, 5 to 300%, 10 to 300%, 20 to 300%, 20 to 200%, 20 to 150%, 20 to 100%, 50 to 100%, 50 to 150% or 50 to 200%. Preferably, the increase occurs over at least one square centimetre of the subject's skin. If the subject suffers from hair loss, generally the change in number of active hair follicles is measured in the area where the hair loss has occurred/is occurring. Higher percentage increases are expected where there is severe hair loss, for example, where a subject has only one hair per square centimetre or several hairs per square centimetres. Optionally, the increase in the number of active hair follicles has occurred at about four months of treatment or at about three months of treatment. In alternate embodiments, the increase in the number of active hair follicles has occurred at about six months or about ten months of treatment.

Prior to the administration of zinc thymulin, the subject optionally has 0 to 300, 0 to 200, 0 to 100, 10 to 100, 0 to 50, 10 to 50, 0 to 20 or 10 to 20 hairs per square centimetre. Subjects with 100 to 300 hairs per square centimetre still have a normal amount of hair (ie normal hair density). Subjects with hair loss optionally have 0 to 100, 0 to 50, 0 to 20, 0 to 10, 10 to 60, 10 to 40, 10 to 20, 10 to 30 or 20 to 60 hairs per square centimetre.

Where the subject has a normal hair density, the administration of the zinc thymulin optionally results in a 5 to 100%, 5 to 50%, 5 to 25% or 5 to 10% increase in active hair follicles. Where the subject suffers from hair loss, the administration of the zinc thymulin optionally results in a 5 to 300%, 20 to 300%, 20 to 200%, 20 to 150%, 20 to 100%, 50 to 100%, 50 to 150% or 50 to 200% increase in active hair follicles. Optionally, the increase in the number of active hair follicles has occurred at about four months of treatment or at about three months of treatment. Alternatively, the increase in the number of active hair follicles has occurred at about six months of treatment or about ten months of treatment.

The density of a subject's hair is optionally classified as absent, vellus, intermediate or terminal in accordance with the HGI hair assessment. Following administration of zinc thymulin subjects with at least one absent hair region optionally have an increase in hair density so that one or more absent hair region classifies as vellus hair or alternatively as intermediate hair. This result optionally occurs after four, six or ten months of treatment.

The administration of zinc thymulin in the methods and uses of the invention may be topical, in particular, topical administration to dermal skin or by injection, for example, by dermal or subcutaneous injection. The compositions and medicaments of the invention are optionally formulated for topical administration or injection. When administration is topical, the zinc thymulin can be formulated as a liquid, a gel, a suspension, a serum, an emulsion or a wax. The topical formulation can be administered as a spray or foam. In one embodiment, the zinc thymulin is formulated for iontophoretic application. In this embodiment, following the topical application of the formulation, mild electric current can be used to deliver the active ingredient into the skin. In another embodiment, the zinc-thymulin is administered via liposomal delivery as a liposomal peptide. Liposomal peptides are used in emulsions such as creams to enhance penetration of a peptide. When zinc thymulin is administered as a liposomal peptide, the zinc thymulin is optionally contained in a liposome or alternatively complexed with a liposome forming ingredient. For example, the phosphatidylcholine-zinc-thymulin complex is optionally used for liposomal delivery of zinc thymulin.

The administration of the compositions and medicaments of the invention is preferably to the scalp. In alternate embodiments, the administration can be to eyebrows, eyelashes, moustaches, beads, chest hair or pubic hair. For example, administration of zinc thymulin is an alternative to pubic hair transplants.

In the methods and uses of the invention, the zinc thymulin is administered to the subject either three times per day, twice per day, once per day, every second day or every third day. Once and twice daily administration are preferred. Optionally, application of zinc-thymulin is multiple times per day, for example, 3, 5, 10, 15 or 20 times per day.

In the methods and uses of the invention, the administration of the compositions and medicaments of the invention continues for at least three months, at least four months, at least six months, at least nine months or at least twelve months.

In one aspect, the present invention provides a composition of zinc thymulin comprising an amount of zinc thymulin effective for activating inactive hair follicles. The composition is preferred to be a topical formulation or an injectable formulation. The thymulin in the zinc thymulin is preferred to be amidated and acetylated. The composition optionally also contain an amount of zinc thymulin effective to reverse hair loss. The optionally also includes at least one pharmaceutically or cosmetically acceptable excipient.

It will be understood that the specific dose level of zinc thymulin for any particular subject depends upon a variety of factors including the severity of their hair loss, the cause of their hair loss, any scalp condition(s) the subject may have.

The compositions and medicaments of the invention optionally include zinc thymulin at an amount of about 0.00001%-0.1% w/v, about 0.00001%-0.01% w/v, about 0.0001% to 0.1% w/v, about 0.0001%-0.001% w/v, about 0.001% to 0.01% w/v or about 0.0005%-0.001% w/v. In preferred embodiments, the zinc thymulin is present at an amount of about 0.0005%. If the formulation is solid or semi-solid, the skilled person is able to convert these w/v dosages to equivalent w/w dosages.

The zinc thymulin in the compositions and medicaments of the invention is optionally 30% w/w or more zinc thymulin to other forms of thymulin. Preferably, the zinc thymulin is 50% w/w or more of the thymulin, 70% w/w or more of the thymulin, 90% w/w or more of the thymulin or 99% w/w or more of the thymulin.

Optionally, the composition comprises an amount of zinc thymulin effective for activating inactive hair follicles, at least one preservative and at least one solvent. Optionally, the solvent is water. Optionally, the at least one preservative comprises three preservatives. Optionally, the three preservatives are benzoic acid, sodium benzoate and potassium sorbate. Optionally, the solvent is suitable for topical administration by spray.

Optionally, the composition comprises an amount of zinc thymulin effective for activating inactive hair follicles and at least one solvent. Optionally, the solvent is physiological saline. Optionally, the formulation further comprises a buffer. Optionally, the solvent is suitable for dermal or subcutaneous injection. Optionally, the composition is injected at a dosage of 5 to 10 microlitres per square centimetre.

Compositions and medicaments of the invention optionally include pharmaceutically or cosmetically acceptable excipients.

In some embodiments of the invention, the subject is male. In alternate embodiments of the invention, the subject is female. The subject is preferred to be a human but may alternatively be a mammal such as a dog, a cat, a horse, a sheep or other mammal.

In some embodiments of the invention, the subject is human and between 35 and 90 years of age. Optionally, the subject is 35 to 45 years of age, 40 to 60 years of age, 60 to 80 years of age or 70 to 90 years of age.

In some embodiments of the invention, the subject is human and has suffered hair loss for 3 to 40 years. Optionally, the subject has suffered hair loss for 3 to 5 years, 5-10 years, 10-20 years or 20-40 years.

The zinc thymulin of the invention is optionally administered concurrently with a second pharmaceutically or cosmetically active ingredient. The zinc thymulin of the invention is optionally administered concurrently with minoxidil, finasteride, dutasteride or a combination of two or more of these.

Any composition or medicament of the invention optionally further comprises a second active ingredient. The second active ingredient could be a vitamin, herbal extract, amino acid, peptide or other cosmetic or therapeutic active. The second active ingredient could improve the rate of hair growth, the diameter or thickness of each hair, the pigmentation of the hair, be an anti-inflammatory, provide nutrition to the hair or provide another benefit. Examples of active ingredients that could be included in the composition are proline, cysteine, lysine, phenylalanine, vitamin B9 (ie folic acid and its activated form of folinic acid), vitamin B3, vitamin D3, coenzyme 010, annexin, keratin peptide EEINEINR, thymulin per se, keratin peptide RIIEGEEHR, β-catenin, LKKTETQ peptide, fallopia japonica, epimedium, rubus idaeus, phosphatidylcholine, hyaluronic acid, thyrotropin and neurovascular peptides called substance P peptide, PACAP (pituitary adenylate cyclase-activating polypeptide) and vasoactive intestinal peptide, plant derived polyphenols and flavonoids of apigenin, icariin, kaempferol, baicalin, chlorogenic acid, caffeic acid, quercetin and epigallocatechin gallate.

In one aspect, the method for activating inactive hair follicles comprises the steps of (i) identifying a subject that is in need of (or desires to) increase the number of active hair follicles; and (ii) administering to the subject an effective amount of zinc thymulin, thereby activating inactive hair follicles.

In another aspect, the method for treating hair loss comprises the steps of (i) identifying a subject suffering from hair loss; and (ii) administering to the subject in need thereof an effective amount of zinc thymulin, thereby treating the hair loss and preferably reversing the hair loss.

In one aspect, the present invention also provides a novel synthetic form of zinc-thymulin, wherein the Ser⁴-OH, Ser⁸-OH and Asn⁹-CO₂ are coordinated with the zinc ion, the Asn⁹ is amidated and the pyrGlu¹ is acetylated.

The present invention has a number of specific forms. Additional embodiments of these forms are as discussed elsewhere in the specification. The following aspects of the invention further describe options for administration of the ODT. For example, in a further aspect the present invention provides a method of activating inactive hair follicles comprising administering a composition to a subject, wherein the composition comprises an effective amount of exogenous zinc thymulin and wherein a specific region of hair of at least one centimetre squared has improved by at least one category according to the HGI hair assessment, for example, the area has progressed from absent to vellus or intermediate or from vellus to intermediate. Optionally, the improvement occurs following six months of administration of zinc thymulin. Optionally, the administration is daily or twice daily.

In a further aspect the present invention provides a method of activating inactive hair follicles comprising administering a composition to a subject, wherein the composition comprises an effective amount of exogenous zinc thymulin and wherein a specific region of hair of at least one centimetre squared has improved by at least 1, 2 or 3 units according to the VAS hair assessment. Optionally, the improvement in the VAS hair assessment is statistically significant in a plurality of subjects that have received daily or twice daily administration of an effective amount of zinc thymulin for 4, 6 or 10 months.

In a further aspect the present invention provides a method of activating inactive hair follicles comprising administering a composition to a subject, wherein the composition comprises an effective amount of exogenous zinc thymulin and wherein the subject suffers from hair loss. Preferably, the hair loss is reversed by generating new hair growth.

In a further aspect the present invention provides a method of activating inactive hair follicles comprising administering a composition to a subject, wherein the composition comprises an effective amount of exogenous zinc thymulin and wherein the subject has normal hair density.

In a further aspect the present invention provides a method of activating inactive hair follicles comprising administering a composition to a subject, wherein the composition comprises an effective amount of exogenous zinc thymulin and wherein the method further increases the number of hair follicles producing hair with natural pigment.

In a further aspect the present invention provides a method of activating inactive hair follicles comprising administering a composition to a subject, wherein the composition comprises an effective amount of exogenous zinc thymulin and wherein a the number of active hair follicles in at least one square centimetre of the subject's hair increases by 5 to 5,000%.

In a further aspect the present invention provides a method of activating inactive hair follicles comprising administering a composition to a subject, wherein the composition comprises an effective amount of exogenous zinc thymulin, wherein the subject has normal hair density and wherein the number of active hair follicles in at least one square centimetre of the subject's hair increases by 5 to 100%.

In a further aspect the present invention provides a method of activating inactive hair follicles comprising administering a composition to a subject, wherein the composition comprises 0.00001%-0.1% w/v exogenous zinc thymulin.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Broad image of the scalp of subject 4 of the clinical trial described in Example 3 before treatment with 0.0005% zinc thymulin topical spray. Camera: Samsung Galaxy Note 4 camera with settings F2.2, focal length 31 mm, LED flash.

FIG. 2: Broad image of subject 4 of the clinical trial described in Example 3 after 3 months twice daily treatment with 0.0005% zinc thymulin topical spray. Camera: Samsung Galaxy Note 4 camera with settings F2.2, focal length 31 mm, LED flash.

FIG. 3: Close-up image of subject 4 of the clinical trial described in Example 3 before treatment before treatment with 0.0005% zinc thymulin topical spray. Camera: Samsung Galaxy Note 4 camera with settings F2.2, focal length 31 mm, LED flash.

FIG. 4: Close-up image of subject 4 of the clinical trial described in Example 3 after 3 months twice daily treatment with 0.0005% zinc thymulin topical spray. Camera: Samsung Galaxy Note 4 camera with settings F2.2, focal length 31 mm, LED flash.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have identified a novel composition for and method of improving hair growth comprising administration of zinc thymulin. Thymulin is a nonapeptide with the amino-acid sequence pyrGlu-Ala-Lys-Ser-Gln-Gly-Gly-Ser-Asn.

As used herein, ‘thymulin’ includes any peptide with the sequence pyrGlu-Ala-Lys-Ser-Gln-Gly-Gly-Ser-Asn including synthetic thymulin, isolated thymulin, acetylated thymulin, amidated thymulin, acetylated and amidated thymulin, other forms of thymulin and other derivatives of thymulin (that do not alter the amino acid sequence).

As used herein, ‘zinc thymulin’ includes any zinc complex with any form of thymulin including the form of zinc thymulin wherein a single zinc atom is complexed to the two serine amino acids and the asparagine amino acid.

The phrase ‘effective amount’ generally refers to either a therapeutically effective amount or a cosmetically effective amount of zinc thymulin.

The phrase ‘normal hair density’ generally refers to a density of between 100-300 follicles per square centimetre.

The words ‘treat’ or ‘treatment’ refer to therapeutic treatment wherein the object is to lessen the impact of or reverse a physiological disease or disorder or its symptoms. In the context of this invention, the disease, disorder or symptom thereof may be a form of hair loss.

The word ‘cosmetic’ refers to compositions or methods for altering appearance or maintaining the condition of an external part of the body. Altering appearance could include altering visual appearance, feel or odour. In the context of this invention, a cosmetic may alter the appearance or maintain the appearance of hair, for example, hair quantity, volume or density. Improvements in appearance of the hair include visibly detectable improvements.

The words ‘prevent’ and ‘prevention’ generally refer to prophylactic or preventative measures for protecting or precluding an individual not having a given disease or disorder from progressing to that disease or disorder or not having an undesirable cosmetic appearance from progressing to have that undesirable cosmetic appearance.

Although this invention has been applied in humans, the invention is also useful for therapeutic and/or cosmetic veterinary purposes, for example, to improve hair growth in horses, dogs, cats or other animals.

The phrases ‘pharmaceutically acceptable’ and ‘cosmetically acceptable’ indicate that the substance or composition must be compatible chemically and/or toxicologically, with the other ingredients in the composition or formulation, and/or the mammal being treated therewith.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), hydrocarbon-based materials such as microsponges and polymer matrices, polysaccharides such as cellulose and its derivatives, and polyacrylic acids.

A topical composition optionally includes one or more components adapted to improve the stability or effectiveness of the applied formulation, such as stabilizing agents, suspending agents, emulsifying agents, viscosity adjusters, gelling agents, buffers, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation is optionally prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Creams and lotions are often similar to one another, differing mainly in their viscosity. Both lotions and creams are optionally opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, optionally also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the final product.

Emulsifiers for use in topical formulations include, but are not limited to, ionic emulsifiers and non-ionic emulsifiers like cetearyl alcohol, phosphatidylcholine (lecithin), polyoxyethylene oleyl ether, acacia gum, PEG-40 stearate, PEG-100 stearate, ceteareth-12, ceteareth-20, ceteareth-30, ceteareth alcohol, acrylic acid, acryl acrylate and glyceryl stearate.

Suitable viscosity adjusting agents include, but are not limited to, protective colloids or nonionic gums such as hydroxyethylcellulose, xanthan gum, carrageenan, magnesium aluminum silicate, silica, microcrystalline wax, beeswax, paraffin, and cetyl palmitate.

A topical gel composition is optionally formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer, ammoniated glycyrrhizinate or other gel/scaffold compounds of alginate, alginic acid, polylactic acid or polyglycolic acid.

Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate are optionally used within topical formulations.

Suitable buffers include, but are not limited to, sodium bicarbonate, sodium phosphate, disodium phosphate, citric acid and calcium carbonate. Sodium bicarbonate is a preferred buffer for formulations of zinc thymulin.

Preservatives include, but are not limited to, antimicrobials such as methylparaben, propylparaben, sorbic acid, benzoic acid, potassium sorbate, sodium benzoate, caprylic acid and formaldehyde, as well as physical stabilizers and antioxidants such as vitamin E, sodium ascorbate/ascorbic acid and propyl gallate.

Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils.

Other suitable additional ingredients that are optionally included in a topical formulation include, but are not limited to, abrasives, absorbents, anticaking agents, antifoaming agents, antistatic agents, astringents (such as witch hazel), alcohol and herbal extracts such as chamomile extract, binders/excipients, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions are optionally formulated for transdermal administration or for dermal administration, that is, delivery into the skin rather than through the skin.

Formulations for administration by injection include liquids, dispersions, such as suspensions or emulsions, formulations containing peptide scaffolds, polyglycolic and polylactic threads and matrices, dissolving microneedles, nanoparticles or alginate and alginic acid. These include solvents (in particular physiological saline solvents), chelating agents (such as EDTA and citric acid), solubilisers, preservatives (such as benzyl alcohol, phenol, sorbic acid, parabens and chlorocresol).

Formulations for administration by injection may be presented as a solid powder or a freeze dried powder ready for reconstitution with a liquid before administration.

The compositions of the invention optionally include one or more additional active therapeutic or cosmetic ingredients. Suitable ingredients include 3-catenin, LKKTETQ peptide, EEINEINR peptide, RIIEGEEHR peptide, substance P peptide, PACAP peptide, vasoactive intestinal peptide, anti-inflammatory agents, vitamins, antioxidants, amino acids, herbal extracts including polyphenols and flavonoids, hair strengthening agents, temporary hair volumising agents and temporary hair thickening agents.

β-catenin optionally induces hair growth. A peptide with the sequence LKKTETQ (leucine-leucine-lysine-threonine-glutamic acid-threonine-glutamine), which is the actin-binding site with an additional peptide Q, promotes hair growth from existing active hair follicles when topically applied to rodent skin. EEINEINR and RIIEGEEHR are both keratin peptides useful for improving the thickness of the hair shaft. These peptides are optionally used in their native form, a synthetic form or an amidated and/or acetylated form. Anti-inflammatory agents can be used to reduce the inflammation often present during hair loss suitable examples include, but are not limited to, Annexin. Suitable vitamins include, but are not limited to, vitamin B9 and vitamin B3. Suitable herbal extracts include, but are not limited to, Fallopia japonica, epimedium and rubus idaeus. Suitable antioxidants include, but are not limited to, coenzyme Q10. Hair strengthening agents include, but are not limited to, keratin, wheat or oat proteins that reinforce the hair.

### Example 1—Synthesis of Zinc Thymulln

A. Preparation of Thymulin

Thymulin has the amino acid sequence: pyrglutamic acid-alanine-lysine-serine-glutamine-glycine-glycine-serine-asparagine, H-PyrGlu-Ala-Lys-Ser-Gln-Gly-Gly-Ser-Asn-OH. Thymulin peptide was prepared using F-moc peptide protocols.

Materials: Amino acids (and protecting groups): PyrGlu (OtBu: t-butyl ester), Ala, Lys (Boc: t-butyloxycarbonyl), Ser (tBu: t-butyl ether), Gin (Trt: trityl or triphenylmethyl), Gly, Asn (Trt: trityl or triphenylmethyl) and Asn (Trt: trityl or triphenylmethyl).

Resin: Rink amide resin.

Reagents: piperidine, N,N-dimethylformamide (DMF), N-methylpyrrolidone (NMP), 2-(1H-benzotriazol-1-yl)-1,1,3,3-tetramethyluronium hexafluorophosphate (HBTU-coupling agent), N,N-Diisopropylethylamine (DIEA) and dichloromethane (DCM).

Synthesis for each amino acid cycle (under nitrogen gas):
1. Swell resin and prewash with DMF for 15 minutes
2. Deprotection with 20% piperidine in DMF
3. Coupling with 0.4M HBTU/DMF, 2M DIEA/NMP
4. Capping with DMF

Cleavage: removal of DMF from resin with dichloromethane, cleavage cocktail of trifluoroacetic acid 90%, thioanisole 5%, EDT (1,2-ethanedithiol) 3%, anisole 2%; 1 ml per 100 mg resin; incubation for 3 hours. Peptide precipitation (amidated form precipitated from Rink amide resin) with cold diethyl ether and centrifugation, repeated three times.

B. Modification of Thymulin

The peptide prepared by the above F-moc protocol is modified by N terminal acetylation and C terminal amidation to improve stability and make the functionality similar to that of the native peptide. Following these modifications, the thymulin has the following structure:

H₃CC(O)-Pyr-Glu-Ala-Lys-Ser-Gln-Gly-Gly-Ser-Asn-NH₂

The skilled person understands how to make these modifications. The example in A above precipitates the amidated form. One method of acetylation is reconstitution in 10% glacial acetic acid, freeze at minus (−) 80 degrees ° C. and lyophilisation; repeated three times. Modified thymulin was stored minus at (−) 20 degrees ° C.

C. Preparation of Zinc Thymulin

The synthesized amidated and acetylated form of thymulin was reacted with zinc oxide compound to yield zinc thymulin. In the reaction, the ratio is by molar mass: 1M thymulin:3M ZnO or its proportional equivalent of millimolar, micromolar or nanomolar mass in 10% glacial acetic acid at 20 degrees ° C. Zinc oxide is dissolved in 10% glacial acetic acid at 20 degrees ° C. and the synthesized thymulin is added. The solution is stirred for 1 hour. Following completion of the reaction the reaction mixture is frozen at minus (−) 80 degrees ° C. and lyophilized in a freeze-dryer to yield the final composite product of the active zinc-thymulin (MW 924.23) and zinc acetate (MW 183.48) powder.

### Example 2—Preparation of Zinc Thymulin for Topical Application

A topical formulation containing 0.0005% zinc thymulin was prepared as a liquid hair spray was prepared according to Table 1.

TABLE 1

| Ingredient | CAS number | % concentration | Function |
|---|---|---|---|
| Zinc-thymulin | 63958-90-7 | 0.0005% | Active |
| (to activate | and 1314-13-2 | | |
| hair follicle | | | |
| regeneration) | | | |
| Benzoic acid | 65-85-0 | 0.05% | Preservative |
| Sodium | 532-32-1 | 0.08% | Preservative |
| benzoate | | | |
| Potassium | 24634-61-5 | 0.12% | Preservative |
| sorbate | | | |
| Distilled water | — | To 100% | Solvent |

The analytical grade benzoic acid, sodium benzoate and potassium sorbate in table 1 were weighed and then dissolved in distilled water with sonication for 15 minutes at 20° C.

The zinc thymulin prepared according to the method set out in Example 1, was weighed, added to the formulation and sonicated for 5 minutes. The formulation was decanted into spray bottles at 20° C. Spray bottles were stored at 4° C.

The above formulation has also been prepared with an additional ingredient, 0.00005% amidated & acetylated LKKTETQ peptide (molecular weight 846.48). Natural form LKKTETQ peptide has been reported to promote growth of hair from active hair follicles but not to activate inactive hair follicles.

When included in the formulation the LKKTETQ peptide is weighed and added to the formulation at the same time as the zinc thymulin is added and sonicated for 5 minutes along with the zinc thymulin.

### Example 3—Clinical Trial Using Topical Zinc Thymulin

Human subjects suffering from hair loss had a marker identified near their hair loss, such as a freckle or mole. The area near the marker affected by hair loss was photographed using a Samsung Galaxy Note 4 camera with settings F2.2, focal length 31 mm, LED flash and the number of active hair follicles in a set area were quantified.

The aqueous spray formulation prepared in Example 2 was administered topically to the scalp of the trial subjects twice daily to the hair loss region(s) of the scalp. The formulation used for subjects 1, 2 and 3 contained 0.00005% LKKTETQ peptide (molecular weight 846.48). The formulation used by subject 4 did not include LKKTETQ peptide.

At 16 weeks for subject 1 and 12 weeks for subjects 2, 3 and 4, the number of active hair follicles in the set area was quantified again. Multiple set areas of hair loss were evaluated for trial subjects 1, 2 and 3. These areas are designated “a” and “b” in Table 2 and Table 3. Table 1 provides information on each subject before treatment. Table 3 provides information on each subject after treatment. Table 4 provides the results as percentage increase in active hair follicles.

TABLE 2
Active hair follicles per square centimetre before treatment

| | | | Number of |
|---|---|---|---|
| | | Number of | active hair |
| | | active hair | follicles before |
| | Size of subject | follicles before | treatment per |
| Subject area | area (cm²) | treatment | cm² |
| 1a | 1.96 | 31 | 16 |
| 1b | 1.69 | 30 | 18 |
| 2a | 0.6 | 34 | 57 |
| 2b | 1.21 | 217 | 179 |
| 3a | 1.26 | 13 | 10 |
| 3b | 3.6 | 26 | 7 |
| 4 | 1.04 | 17 | 16 |

TABLE 3
Active hair follicles per square centimetre after treatment

| | | | Number of |
|---|---|---|---|
| | | Number of | active hair |
| | | active hair | follicles after |
| | Size of subject | follicles after | treatment per |
| Subject area | area (cm²) | treatment | cm² |
| 1a | 1.96 | 48 | 24 |
| 1b | 1.69 | 36 | 21 |
| 2a | 0.6 | 76 | 127 |
| 2b | 1.21 | 230 | 191 |
| 3a | 1.26 | 39 | 31 |
| 3b | 3.6 | 32 | 9 |
| 4 | 1.04 | 40 | 38 |

TABLE 4
percentage increase in active hair follicles

| | Number of | Number of | Percentage |
|---|---|---|---|
| | active hair | active hair | increase in |
| | follicles before | follicles after | active hair |
| Subject area | treatment | treatment | follicles |
| 1a | 31 | 48 | 55 |
| 1b | 30 | 36 | 20 |
| 2a | 34 | 76 | 124 |
| 2b | 217 | 230 | 6 |
| 3a | 13 | 39 | 200 |
| 3b | 26 | 32 | 23 |
| 4 | 17 | 40 | 135 |
| Range | | | 6-200 |
| Average | | | 80 |
| increase | | | |

Subject 1 was a 48 year old female with 3 years of hair loss. Subject 2 was a 49 year old male with 3 years of hair loss. Subject 3 was a 43 year old male with 6 years of hair loss. Subject 4 was a 60 year old male with 11 years of hair loss.

Area 2b had an equivalent of 179 active hair follicles per square centimetre prior to treatment. This is within the normal range (ie 100 to 300 hairs per square centimetre) and is an example of an increase in active hair follicles when zinc thymulin is administered to an area of a subject that has a normal quantity of hair.

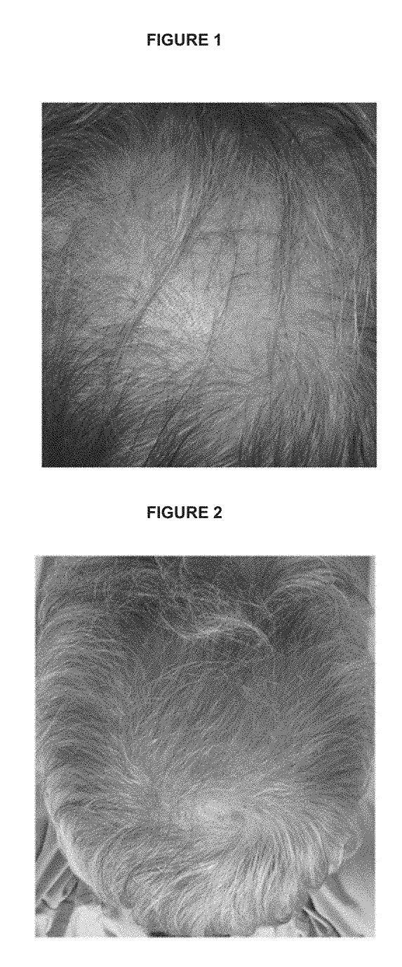

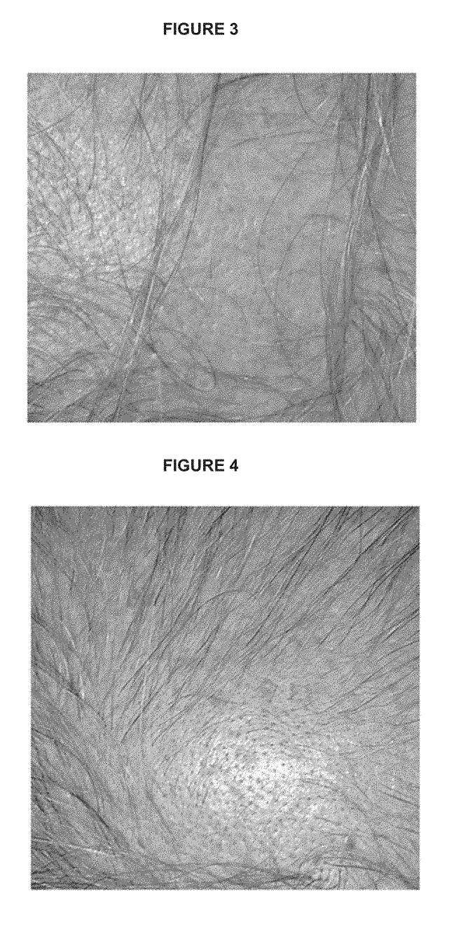

Low resolution and high resolution images of Subject 4 before treatment are in FIG. 1 and FIG. 3 respectively and low resolution and high resolution images of Subject 4 after treatment are included in FIG. 2 and FIG. 4, respectively.

### Example 4—Injectable Formulation of Zinc Thymulin

0.0005% zinc thymulin is dissolved in a solvent of normal physiological saline.

The formulation is optionally buffered, for example with phosphate so that the solvent is phosphate buffered saline. This liquid formulation is suitable for injection into the dermis and/or subdermis at a dosage of 5 to 10 microlitres per square centimetre.

Additional supporting active peptides and or other active ingredients are optionally added as discussed above.

### Example 5—Further Clinical Trial Using Topical Zinc Thymulin

Methods:

Eighteen consecutive adult subjects were recruited, 17 males and 1 female, age range 35-90 years (mean 55.4, SD 13.3) with a diagnosis of AGA, Norwood classification 2-7, and hair loss duration range of 3-40 years (mean 15.8, SD 9.6). The trial duration for each subject ranged from 4-10 months. The test compound ZT was synthesized by standard Fmoc peptide protocols and administered in water based topical spray to the scalp. Baseline and after treatment images for hair growth were graded by two blinded assessors using two validated scales: 1. numerical visual analog scale (VAS) for global assessment 2. hair growth index (HGI) of images under higher magnification for percentage changes of vellus, intermediate and terminal hair.

Results:

ZT demonstrated no adverse systemic effects or local side effects of redness or scalp irritation in any subject over a total of 3,300 treatment days. Three subjects who were concurrently using minoxidil (N=2) and minoxidil/finasteride (N=1) did not report any drug interaction with ZT. VAS hair assessment improvement was significant in subjects who completed 6 months of treatment (P=0.045, t-test). HGI assessment showed a significant increase in the number of newly observed intermediate hairs in previous “absent hair” regions (P<0.0001) with an average increase of vellus type (32%) and intermediate type (23%) hairs at 6 months. Melanogenesis was observed in several subjects.

Conclusion:

Topical applications of ZT demonstrated safety and established efficacy for initiating and maintaining anagen to treat male pattern baldness when applied for >6 months.

Detail

Subjects

Male and female adult subjects with a diagnosis of AGA were invited to participate in the study. Each subject provided written informed consent except the 90 year old subject who gave verbal consent as he was unable to provide written consent due to arthritis of the hand. The study was conducted in compliance with the Declaration of Helsinki, International Conference on Harmonization Good Clinical Practice guidelines. Eighteen consecutive adult subjects were recruited (17 males and 1 female) with an age range 35-90 years (mean 55.4, SD 13.3) with a diagnosis of AGA Norwood-Hamilton classification 2-7, and with a hair loss history ranging from 3-40 years (mean 15.8, SD 9.6). Entry time points of the volunteers occurred over 6 months with the trial completed at the same endpoint resulting in duration of treatment for subjects in the trial ranging from 4-10 months.

Materials

Thymulin has an amino acid sequence pyrGlu-Ala-Lys-Ser-Gln-Gly-Gly-Ser-Asn (one letter sequence pEAKSQGGSN) and was synthesized using standard Fmoc protocols on a peptide synthesizer (AAPPTEC Corp. Louisville, Ky., USA). Amino acids, resin, reagents and resin cleavage chemicals were analytical grade (AAPPTEC; Labsupply, Sydney, Australia). Confirmation and purity of thymulin was performed by liquid chromatography coupled to an electrospray mass spectrometer (LCMS). The LCMS was a Shimadzu 2010AD system.

Purity of the peptide was established at >95% with LC conditions employing reverse phase chromatography with mobile phase A 0.1% acetic acid in MilliQ water and phase B of analytical grade methanol, gradient 0-50% B over 30 minutes, using a 2.1 mm×150 mm C8 column (Grace Vydac). The operating conditions of the MS were 1.5 kV detector voltage in positive ion mode, heat block and CDL temperature at 200 C, 1.5 L/min nitrogen nebulizing gas, m/z scan range 200-1000.

The mass spectra of the eluting peak showed two m/z values of 430 and 375. At the stated MS operating conditions m/z 430 represents the thymulin peptide (mass 858.85) that displayed the [M+2H]2+ state i.e. 429×2 (double electron charge)+1 proton from acetic acid=observed mass 859. The m/z 375 is an expected but variable phase between pyruvate and the amino acids glutamic acid and glutamine that occurs uniquely under electrospray kV detector conditions.

After peptide synthesis the residual trifluoroacetic acid and resin cleavage chemicals were removed by lyophilization with 10% acetic acid, repeated three times. Attachment of the zinc ion was performed by mixing thymulin with zinc oxide (1:3 w/w) in 10% acetic acid and lyophilized to yield ZT. The compound was then dissolved in distilled water with preservatives benzoic acid, sodium benzoate and potassium sorbate.

Citric acid and sodium bicarbonate were used as buffers that resulted in a colorless and odorless solution at pH 5.4. The product given to subjects was a 100 ml spray bottle containing 0.0005% ZT as the active test agent. Subjects were instructed to spray 1-2 ml of solution, twice daily, and rub into the scalp. The spray bottle solution was to be kept away from direct sunlight and heat. Subjects were resupplied with fresh solutions every 6-8 weeks during the trial.

Hair Growth Assessment

Images were captured on a Samsung Galaxy Note 4 phone in high resolution (16 MB image) with flash, F 2.2 and focal length 31 mm. For assessment, baseline and after treatment images were de-identified, paired and randomly allocated with respect to left/right sides. Images were presented for analysis to assessors in Powerpoint format files on a 55 inch high resolution television screen. All images and original assessor scorecards were archived in PDF format for external reviewer confirmation.

Two validated assessment methods were used to grade the changes:

1. Numbered visual analog scale (VAS) where the assessor gives a value between 0-10 of the image (0=total absence of hair, 10=full abundant hair) (Price, 2008).

2. Hair Growth Index (HGI) that is a more in-depth scale of analysis where higher magnification of images can identify the four types of hair regrowth that are designated as absent, vellus, intermediate or terminal (Bernado, 2003). For each image these four growth stages are given percentage values that added together give a raw score total of 100%. Each percentage value for absent, vellus, intermediate, and terminal are multiplied by 0,1,2,3 respectively to yield a final adjusted score ranging from a possible minimum score of 0 (total absence of hair) to a maximum score of 300 (full terminal hair). Scores were pooled for paired t-test, and P<0.05 was established for significance.

Assessment was graded by two independent assessors in a single blind manner. Assessors underwent three training sessions over two weeks prior to the final evaluation. Reliability and validation of the assessors using the scoring methods was conducted by a test/retest at two weeks with Pearson's R. Results showed good intrasubject reliability at two weeks (assessor #1 R=0.69, assessor #2 R=0.71, respectively); and similarly, intersubject reliability had improved to a high level at two weeks (first training session R=0.54, third training session at two weeks R=0.76).

Results

Demographic data of the 18 subjects and their individual trial duration of ZT use are shown in Table 5. The VAS analysis showed no significant growth in the total group (P=0.07) at the end of the trial but significant growth was observed in the 11 subjects who had completed at least 6 months of treatment (P=0.045) (Table 6).

TABLE 5
Demographic data of subjects and trial duration

| | | | | | | zinc- |
|---|---|---|---|---|---|---|
| | | | hair | | other | thymulin |
| sub- | | age | loss | Norwood | current | use |
| ject | gender | (years) | (years) | scale | treatments | (months) |
| 1 | male | 35 | 10 | 2 | | 9 |
| 2 | female | 49 | 3 | 2 | | 8 |
| 3 | male | 64 | 5 | 4 | minoxidil | 7 |
| 4 | male | 61 | 11 | 3 vertex | | 4 |
| 5 | male | 53 | 30 | 5A | | 6 |
| 6 | male | 69 | 15 | 5A | | 6 |
| 1 | male | 55 | 15 | 6 | | 9 |
| 8 | male | 44 | 15 | 2A | finasteride & | 6 |
| | | | | | minoxidil | |
| 9 | male | 35 | 15 | 5A | | 4 |
| 10 | male | 49 | 10 | 7 | minoxidil | 5 |
| 11 | male | 64 | 5 | 3 vertex | | 5 |
| 12 | male | 68 | 5 | 6 | | 6 |
| 13 | male | 90 | 40 | 7 | | 5 |
| 14 | male | 55 | 20 | 7 | | 4 |
| 15 | male | 50 | 10 | 2A | | 5 |
| 16 | male | 44 | 20 | 7 | | 6 |
| 17 | male | 50 | 30 | 4 | | 10 |
| 18 | male | 60 | 15 | 5A | | 6 |

TABLE 6
VAS (0-10) of each subject by the two assessors (#1 and #2)
[P = 0.07 for poo led results, P = 0.045* (t-test) for subjects
using zinc-thymulin for >6 months].

| | | | VAS after |
|---|---|---|---|
| | subject | VAS baseline | treatment |
| Assessor #1 | 1 | 7 | 8 |
| | 2 | 7 | 8 |
| | 3 | 8 | 6 |
| | 4 | 6 | 8 |
| | 5 | 9 | 8 |
| | 6 | 7 | 8 |
| | 7 | 8 | 6 |
| | 8 | 8 | 9 |
| | 9 | 9 | 8 |
| | 10 | 6 | 7 |
| | 11 | 9 | 8 |
| | 12 | 7 | 8 |
| | 13 | 5 | 8 |
| | 14 | 7 | 6 |
| | 15 | 7 | 8 |
| | 16 | 6 | 7 |
| | 17 | 8 | 9 |
| | 18 | 8 | 9 |
| Assessor #2 | 1 | 7 | 6 |
| | 2 | 7 | 8 |
| | 3 | 7 | 6 |
| | 4 | 6 | 8 |
| | 5 | 6 | 7 |
| | 6 | 7 | 6 |
| | 7 | 5 | 4 |
| | 8 | 8 | 9 |
| | 9 | 6 | 7 |
| | 10 | 4 | 5 |
| | 11 | 8 | 7 |
| | 12 | 6 | 8 |
| | 13 | 5 | 8 |
| | 14 | 7 | 6 |
| | 15 | 7 | 6 |
| | 16 | 4 | 5 |
| | 17 | 7 | 8 |
| | 18 | 8 | 9 |

In 10 subjects the images could be magnified with acceptable clarity and definition to perform the HGI analysis. In the raw score analysis (Table 7) there was significantly less “absent hair” after treatment (P=0.008) and a significant increase of intermediate hair in the baseline/after treatment category (P=0.02). In the HGI adjusted scores (Table 8) there was confirmation of the increase of intermediate hair when compared for the baseline/after treatment category (P=0.03).

Only one side effect was reported in the entire study from a nonactive constituent. Subject #1 used the topical spray each day for nine months and reported a single transient episode of one hour of redness to the forehead after exposure to the sun for several hours, followed by a facial forehead exfoliative scrub cream and then application of the spray.

The investigator (ERV, a maxillofacial surgeon) considered that the redness was due to the action of the citric acid buffer on the combined sun-irradiated and abraded skin where it mimicked a low grade cosmetic facial chemical peel effect.

The subject continued to use the spray with no further recurrence.

TABLE 7
Hair Growth Index analysis of row percentage scores (N = 10 subjects) by each assessor (A#1 and A#2)
The four hair types graded as: A (absent), V (vellus), I (intermediate), T (terminal)
before and after trial completion. P values are shown at the bottom of the table.

| | subject | % A before | % A after | % V before | % V after | % I before | % I after | % T before | % T after |
|---|---|---|---|---|---|---|---|---|---|
| A#1 | 2 | 50 | 20 | 10 | 25 | 10 | 25 | 30 | 30 |
| | 3 | 30 | 20 | 5 | 10 | 5 | 10 | 60 | 60 |
| | 4 | 50 | 40 | 10 | 25 | 20 | 25 | 20 | 10 |
| | 7 | 50 | 50 | 20 | 10 | 20 | 20 | 10 | 20 |
| | 9 | 30 | 40 | 40 | 20 | 10 | 20 | 20 | 30 |
| | 10 | 50 | 50 | 30 | 20 | 10 | 15 | 10 | 15 |
| | 14 | 50 | 50 | 20 | 10 | 10 | 10 | 20 | 30 |
| | 16 | 50 | 40 | 10 | 25 | 10 | 25 | 30 | 10 |
| | 17 | 50 | 40 | 0 | 15 | 20 | 25 | 30 | 20 |
| | 18 | 30 | 15 | 10 | 5 | 20 | 25 | 40 | 55 |
| A#2 | 2 | 40 | 20 | 10 | 30 | 30 | 30 | 20 | 20 |
| | 3 | 40 | 30 | 0 | 20 | 10 | 30 | 50 | 20 |
| | 4 | 40 | 10 | 10 | 30 | 30 | 30 | 20 | 30 |
| | 7 | 20 | 30 | 10 | 0 | 40 | 30 | 30 | 40 |
| | 9 | 10 | 10 | 50 | 40 | 20 | 20 | 20 | 30 |
| | 10 | 50 | 40 | 10 | 20 | 20 | 30 | 20 | 10 |
| | 14 | 40 | 50 | 20 | 10 | 20 | 20 | 20 | 20 |
| | 16 | 50 | 40 | 0 | 30 | 20 | 20 | 30 | 10 |
| | 17 | 30 | 30 | 10 | 20 | 40 | 30 | 20 | 20 |
| | 18 | 30 | 10 | 10 | 10 | 10 | 20 | 50 | 60 |
| P= | | | 0.008* | | 0.17 | | 0.02* | | 0.86 |

TABLE 8
Hair Growth Index analysis of adjusted scores (N = 10 subjects) by each assessor (#1 and #2)
The four types graded as: A (absent), V (vellus), I (intermediate), T (terminal)
before and after trial completion. P values are shown at the bottom of the table.

| | subject | A before | A after | V before | V after | I before | I after | T before | T after |
|---|---|---|---|---|---|---|---|---|---|
| Assessor #1 | 2 | 0 | 0 | 10 | 25 | 20 | 50 | 90 | 90 |
| | 3 | 0 | 0 | 5 | 10 | 10 | 20 | 180 | 180 |
| | 4 | 0 | 0 | 10 | 25 | 40 | 50 | 60 | 30 |
| | 7 | 0 | 0 | 20 | 10 | 40 | 40 | 30 | 60 |
| | 9 | 0 | 0 | 40 | 20 | 20 | 20 | 60 | 90 |
| | 10 | 0 | 0 | 30 | 20 | 10 | 30 | 30 | 45 |
| | 14 | 0 | 0 | 20 | 10 | 20 | 20 | 60 | 90 |
| | 16 | 0 | 0 | 10 | 25 | 20 | 50 | 90 | 30 |
| | 17 | 0 | 0 | 0 | 15 | 40 | 50 | 90 | 60 |
| | 18 | 0 | 0 | 10 | 15 | 40 | 50 | 90 | 60 |
| Assessor #2 | 2 | 0 | 0 | 10 | 30 | 60 | 60 | 60 | 60 |
| | 3 | 0 | 0 | 0 | 20 | 20 | 60 | 150 | 60 |
| | 4 | 0 | 0 | 10 | 30 | 60 | 60 | 60 | 90 |
| | 7 | 0 | 0 | 10 | 0 | 80 | 60 | 90 | 120 |
| | 9 | 0 | 0 | 50 | 40 | 40 | 40 | 60 | 90 |
| | 10 | 0 | 0 | 10 | 20 | 40 | 60 | 60 | 30 |
| | 14 | 0 | 0 | 20 | 10 | 40 | 40 | 60 | 60 |
| | 16 | 0 | 0 | 0 | 30 | 40 | 40 | 90 | 30 |
| | 17 | 0 | 0 | 10 | 20 | 80 | 60 | 60 | 60 |
| | 18 | 0 | 0 | 10 | 10 | 20 | 40 | 150 | 180 |
| P= | | | | | 0.18 | | 0.03* | | 0.52 |

### DISCUSSION

Safety

The ZT topical formulation was shown to be very safe with only one side effect from a non-active constituent reported in 18 test subjects over a total of 111 months (˜3,300 days). Specifically, there was no redness, scalp irritation or deterioration of hair color, quality or quantity. In addition, three subjects were using minoxidil and/or finasteride during the trial with no observable drug interaction suggesting concurrent use of these approved FDA drugs with ZT is possible. The excellent safety profile of ZT demonstrated in this study offers an opportunity for treating people with alopecia where side effects of finasteride and minoxidil may be problematic; depression, suicidal thoughts and persistent sexual problems from finasteride, pruritis and local irritation from minoxidil.

Anagen Initiation

In the transition to observed hair development there was a highly significant increased number of intermediate hairs that had progressed from the absent hair stage (P<0.0001) indicating widespread hair follicle activation. To confirm this effect subject #17 (30 year hair loss) underwent a fluorescence spectroscopy procedure of the scalp. The images showed 6 pre-existing terminal hairs under visible white LED light but spectroscopy revealed an additional 59 developing vellus hairs. Further spectroscopic research is required to confirm this effect but this potentially indicates that extended use of ZT may treat people with Norwood classification 6-7. Supporting evidence was seen when comparing the assessor pooled responses of ‘total absent hair’ where 65% (13/20) of subjects improved.

Melanogenesis

Melanogenesis was clearly observed with the higher magnification images in several older subjects who developed numerous intermediate hairs that were dark and resembled the color of hair when they were younger.

### CONCLUSION

Global visual improvement was statistically significant when ZT was used for >6 months. The study also showed that ZT initiated anagen in AGA subjects with Norwood-Hamilton classification 5-7. Moreover, several subjects were concurrently using finasteride and minoxidil with no evidence of adverse drug interactions with ZT. The compound was seen to induce melanogenesis and suggests further cosmetic applications in addition to its anagen stimulating properties.

### REFERENCES

United States patent publication no. 2011/0281802.
Price V H, Hordinsky M K, Olsen E A. Roberts J L, Siegfried E C, et al. (2008) Subcutaneous efalizumab is not effective in the treatment of alopecia areata. J Am Acad Dermatol 58: 395-402.
Bernardo O, Tang L, Lui H, Shapiro J (2003) Topical nitrogen mustard in the treatment of alopecia areata: A bilateral comparison study. Am Acad Dermatol 2: 291-294.

1.-15. (canceled)

16. A method of activating inactive hair follicles comprising administering a composition to a subject, wherein the composition comprises an effective amount of exogenous zinc thymulin.

17. The method of claim 16 wherein the subject has normal hair density.

18. The method of claim 16 wherein the method treats and/or prevents hair loss.

19. The method of claim 18 wherein the subject suffers from hair loss and the hair loss is reversed.

20. The method of claim 16 wherein the number of active hair follicles in at least 1 square centimetre of the subject's skin is increased by 5 to 300%.

21. The method of claim 18 wherein the number of active hair follicles in at least 1 square centimetre of the subject's skin is increased by 20 to 300%.

22. The method of claim 17 wherein the number of active hair follicles in at least 1 square centimetre of the subject's skin is increased by 5 to 100%.

23. The method of claim 20 wherein the increase has occurred at about 4 months of treatment.

24. The method of claim 21 wherein the increase has occurred at about 4 months of treatment.

25. The method of claim 22 wherein the increase has occurred at about 4 months of treatment.

26. The method of claim 16 wherein the subject's hair density is increased.

27. The method of claim 16 wherein the administration is topical or by injection.

28. The method of claim 16 wherein the zinc thymulin is present at an amount of about 0.0001%-0.1% w/v.

29. The method of claim 16 wherein the zinc thymulin is present at an amount of about 0.001%-0.05% w/v.

30. The method of claim 16 wherein the zinc thymulin is present at an amount of about 0.02% w/v.

31. The method of claim 16 wherein LKKTETQ peptide is also administered to the subject.

32. A method of increasing the number of hair follicles producing hair with natural pigment comprising administering an effective amount of zinc thymulin to a subject.

33. A topical composition comprising about 0.0001%-0.1% w/v zinc thymulin and at least one pharmaceutically or cosmetically acceptable excipient.
